(19) United States
(12) Patent Application Publication
(10) Pub. No.: US 2005/0240169 A1
(43) Pub. Date: Oct. 27, 2005

(54) Apparatus and method for promoting blood circulation to an injury

(76) Inventors: Ronald Kenneth Cardow, Greenwell Point (AU); Wal Johnson, Greenwell Point (AU)

Correspondence Address: Jacobson & Johnson, Suite 285, One West Water Street, St Paul, MN, 55107-2080, US

(21) Appl. No.: 10/518,622

(22) PCT Filed: Jul. 9, 2003
(86) PCT No.: PCT/AU03/00879

§ 371 Date: Dec. 21, 2004

(30) Foreign Application Priority Data
Jul. 10, 2002 (AU) 2002950146

(51) Int. Cl. A61B 18/04

(52) U.S. Cl. 606/27

(57) ABSTRACT

There is an apparatus for promoting or assisting the healing of an injured area (26) of human anatomy by alternative applications of a fluid at differing temperatures. The apparatus includes a spray applicator (23) and a reservoir (1) for the fluid with a pressure pump (14) and fluid heater (11). The apparatus in use is arranged such that the spray applicator (23) is connected to the pressure pump (14) whereby timed sprays of the fluid from the reservoir I at a selected temperature can be applied to the injured area (26) to increase blood circulation.

### FIELD OF THE INVENTION

This invention relates to an apparatus and method for promoting the healing of injuries or other abnormalities which are treatable through increased blood circulation to the affected area.

### BACKGROUND TO THE INVENTION

Injuries to muscles, ligaments and tendons etc. from recreation or work related activities are very common and the cost of these to the community is considerable. While there are a number of established treatments for these conditions such as manipulation, ultrasound and surgery etc. the inventor has found that in many cases these are insufficient or are unduly traumatic for the patient.

### SUMMARY OF THE INVENTION

It is therefore an object of this invention to ameliorate the aforementioned disadvantages or at least provide the public with a practical, non-medical or non-intrusive alternative to known treatments. Accordingly this invention in one aspect discloses an apparatus for promoting or assisting the healing of an area of the human anatomy by alternate application of a fluid at differing temperatures, said apparatus including a spray applicator, a pressure pump means, a reservoir-for containment of said fluid and a fluid heater. wherein in use the apparatus is arranged such that the spray applicator is connected to said pressure pump means and the fluid from said reservoir at a selected temperature is applied for a timed period to said area to promote and increase blood circulation.

Preferably the apparatus further includes a tub or sump for collection and reuse of said fluid after application to said area.

It is further preferred that the fluid reservoir comprise a tank with said heater and pressure pump being built-in together with a thermostat.

It is further preferred that said fluid comprise a solution of salts and minerals dissolved in water.

In another aspect the invention also discloses a method of using the aforementioned apparatus to promote blood circulation to an injured area of the human anatomy which includes the steps of:
selecting differing temperatures for said fluid, and
applying timed sprays of said fluid to the injured area in a manner whereby said sprays alternate between said differing temperatures.

### BRIEF DESCRIPTION OF THE DRAWINGS

One currently preferred embodiment of the invention will now be described with reference to the attached drawings in which:

FIG. 1 is a schematic perspective side view of the fluid tank portion of the apparatus,

FIG. 2 is a plan view of the tank of FIG. 1 with the lid removed,

FIG. 3 is an underside view of the tank,

FIG. 4 is a cross-sectional view of the tank along the lines A-A of FIG. 2, and

FIG. 5 is a pictorial illustration showing apparatus in use.

### DETAILED DESCRIPTION OF THE PREFERRED EMBODIMENT

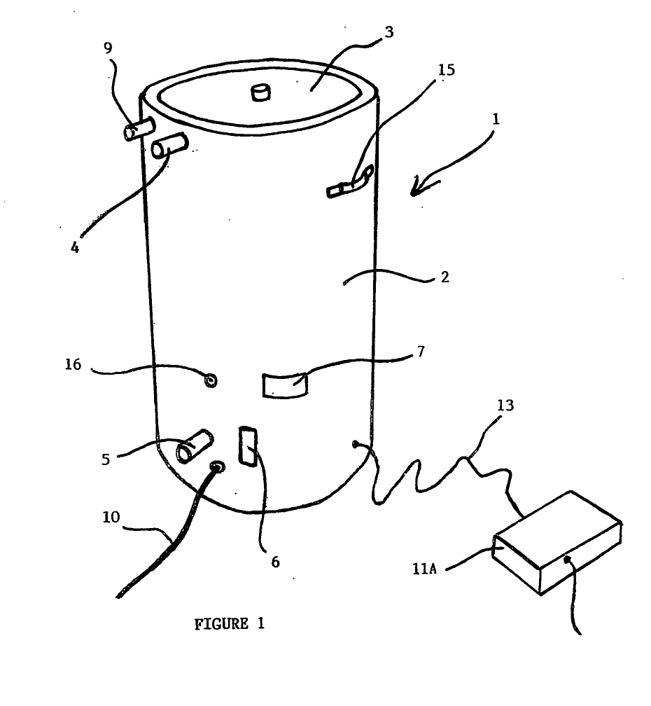

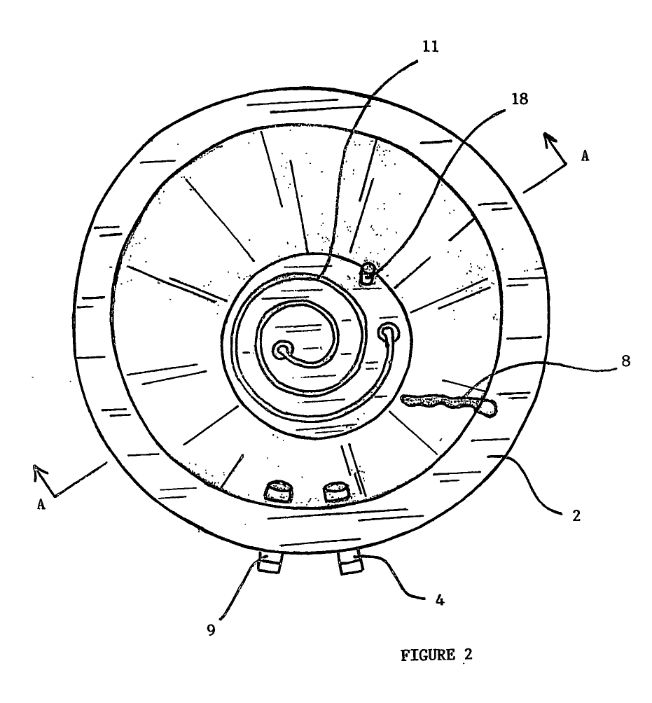

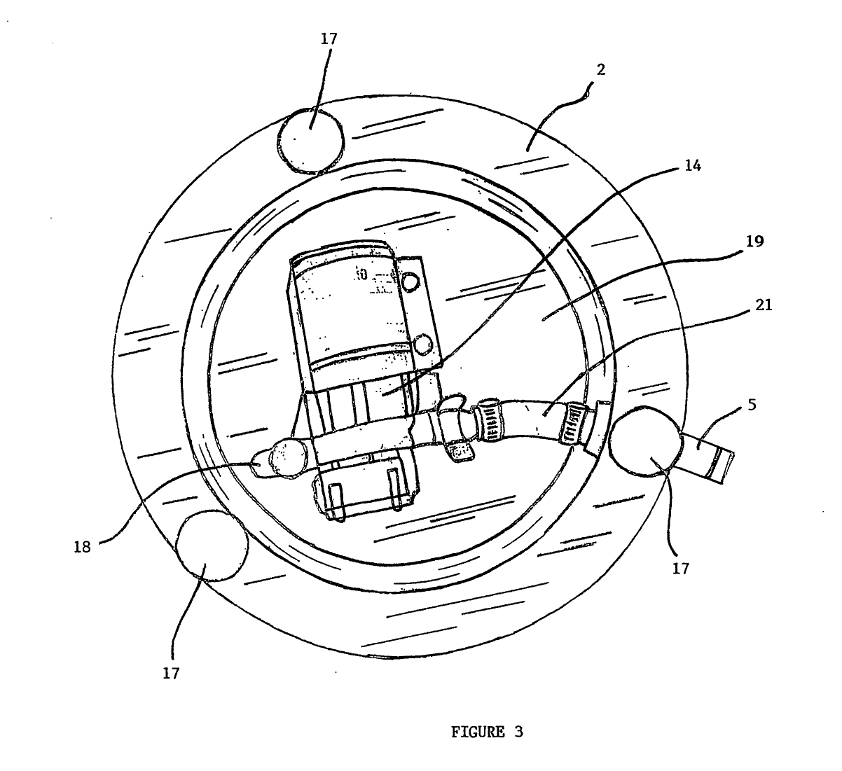

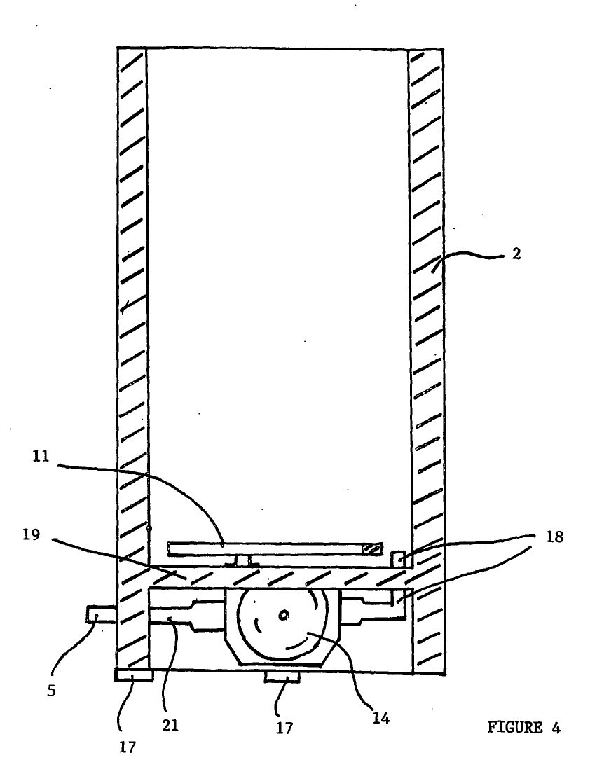

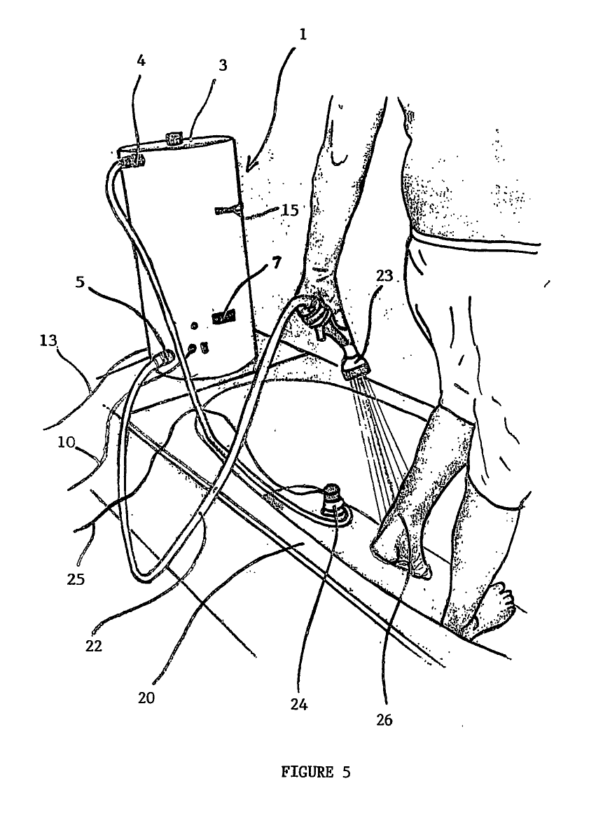

Referring first to FIGS. 1 to 4 there is an upright cylindrical tank 1 constructed of any suitable material such as stainless steel, fibreglass or plastic with heat insulated walls 2 and lid 3. There is a tank inlet 4, an outlet 5. power switch 6, digital thermostat control panel 7, sensor 8 overflow 9, mains power cord 10 for the heating element 11 and 12 volt supply 11A together with power cord 13 for the pump 14. Carry handles 15 may be fitted to each side together with a pilot light 16 for the heater and rubber feet 17. As best shown in FIGS. 3 and 4 there is a pump intake 18 extending up through the recessed bottom 19 of the tank. Although the invention is not limited to any particular capacity for the tank it. may with this embodiment contain about 20 litres of water. The tank is therefore of a suitable size to be placed next to a domestic bath tub 20 as shown in FIG. 5.

The pressure pump 14 is preferably but not essentially operated off the 12 volt supply 11A to avoid electrical shock and is of a commercially available diaphragm type such as that available from Bias Boating of North Parramatta New South Wales Australia under model No. 2088/423/344. Heated water is drawn into the pump through intake 18 at the bottom of the tank which although not shown in the drawings may be fitted with any suitable form of screen or filter. The high pressure outlet side of the pump is connected to the tank outlet 5 through a pipe 21.

In use of the apparatus a pressure hose 22 and six position adjustable spray gun 23 (such as one sold in Australia under the name “Gardeners Choice”) are connected to the tank outlet 5, a sump pump 24 is placed in the bottom of the bathtub 20 for connection to the tank inlet 4 and the mains and 12 volt power cords 10, 13 and 25 for the heating element, pressure pump and sump pump are connected to appropriate electrical supplies.

Although the invention is not limited to the use of any particular type of fluid, with this embodiment a solution of water mixed with additives at the rate of 1 gram/litre is used. Preferably but not essentially these additives may comprise:-

| | |
|---|---|
| Laurel Sulfate | 16% |
| Sodium Metasilicate | 30% |
| Sodium Bicarbonate | 50% |
| Eucalyptus Oil | 2% |
| Tea Tree Oil | 2% |

It has been found that the aforementioned solution of salts and minerals together with the temperature differential created by hot/cold spray cycles causes the blood vessels in the area of the injury to dilate. This increases circulation and promotes healing of the injury.

By way of non-limiting example the thermostat may be set at 41° C. and the spray gun pressure at between 10 and 800 kpa.

An injured area such as for example the user's ankle or lower leg 26 as shown in FIG. 5 is then sprayed with the heated solution for a selected period. The solution as it collects in the tub is returned to the tank by the sump pump 24 for reheating and reuse. Extended spraying is thus possible using only a relatively small amount of solution.

Preferably the heated solution sprays are alternated with cold water sprays at about 10° C. These could be drawn from a separate tank (not shown) or a mains water supply. The number and duration of treatments and the number of hot/cold cycles per treatment would be at the discretion of the user or practitioner operating the apparatus. For a common injury such as a sprained ankle it has been found that the following treatment program using the above solution over a period of two days produces beneficial results.

| Hot Spray Temperature | 41° C. |
|---|---|
| Cold Spray Temperature | 10° C. |
| Hot Spray Duration | 30 minutes |
| Cold Spray Duration | 5 minutes |
| No. Of Hot/Cold Spray Cycles Per Treatment | 1 |
| No. Of Treatments | 6 |

It will thus be appreciated that this invention at least in the form of the embodiment disclosed provides a novel and useful apparatus and method for promoting the healing of damaged tissue in humans. Clearly however the example described is only the currently preferred form of the invention and a wide variety of modifications may be made which would be apparent to a person skilled in the art. For example the size, shape and configuration of the tank, the temperature, pressure and duration of the spray as well as the nature of the additives used may all be changed according to treatment requirements. The invention is also not limited to any specific materials for constructing the tank although stainless steel, fibreglass or plastic are currently preferred.

1. An apparatus for promoting or assisting the healing of an injured area of human anatomy by alternative applications of fluids at differing temperatures, said apparatus including a spray applicator and a reservoir for a first one of said fluids with a pressure pump and fluid heater, the apparatus in use being arranged such that the spray applicator is connected to said pressure pump whereby timed sprays of said first one of said fluids from the reservoir at a selected temperature can be applied to said injured area to increase blood circulation and said apparatus further including a spray source of a second one of said fluids at a temperature below said selected temperature.

2. The apparatus as claimed in claim 1 wherein the fluid reservoir is a tank with said fluid heater and pressure pump being built-in.

3. The apparatus as claimed in claim 2 wherein said apparatus further includes a tub or sump for collection and reuse one or both of said fluids.

4. The apparatus as claimed in claim 3 wherein said first one of said fluids is a solution of salts and minerals dissolved in water.

5. The apparatus as claimed in claim 4 wherein the pressure pump is adapted to operate off a 12 volt supply to avoid electrical shock.

6. The apparatus as claimed in claim 5 and further including a thermostat for controlling said fluid heater.

7. The apparatus as claimed in claim 6 wherein the reservoir is a cylindrical tank of a size suitable for placement in a domestic bathroom.

8. A method of promoting or assisting the healing of an injured area of human anatomy by alternative applications of fluids at differing temperatures, said method including the steps of:-
procuring an apparatus which includes a spray applicator and a reservoir for a first one of said fluids with a pressure pump and fluid heater, the apparatus being arranged such that the spray applicator is connected to said pressure pump wherein timed sprays of said first one of said fluids from the reservoir at a selected temperature can be applied to the injured area to increase blood circulation and said apparatus further including a spray source of a second one of said fluids at a temperature below said selected temperature.
heating said first one of said fluids to the selected temperature in the reservoir, and
applying timed sprays of said first one of said fluids at said selected temperature to the injured area.
alternating said timed sprays with sprays of said second one of said fluids at a temperature below said selected temperature.
